(19) United States
(12) Patent Application Publication
(10) Pub. No.: US 2011/0144592 A1
(43) Pub. Date: Jun. 16, 2011

(54) Haemostatic Valve

(75) Inventors: En Hou Philip Wong, Singapore (SG); Lay Swan Esther Lim, Singapore (SG); Yau Wong, Singapore (SG); Poh Hock Neo, Singapore (SG)

(73) Assignees: SINGAPORE HEALTH SERVICES PTE. LTD., Singapore, SG (SG); NANYANG POLYTECHNIC, Singapore, SG (SG)

(21) Appl. No.: 13/001,404

(22) PCT Filed: Jun. 27, 2008
(86) PCT No.: PCT/SG08/00232

§ 371 Date: Feb. 23, 2011

(51) Int. Cl. A61M 25/04

(52) U.S. Cl. 604/175

(57) ABSTRACT

A haemostatic valve according to an embodiment comprises a valve housing receiving an intravascular device, a first slider arranged on the valve housing moveable in a first direction, and a second slider received in the valve housing moveable in a second direction and elastically biased, the first slider being operatively connected to the second slider via a transmission mechanism so that movement of the first slider towards along the first direction causes the second slider to move away from an elastic valve member along the second direction, and the elastic valve member arranged between and compressible by the second slider, the elastic valve member receiving the intravascular device and compressing the intravascular device when being compressed by the second slider.

The present invention relates to a haemostatic valve.

Conventional haemostatic valves generally utilize a twisting mechanism to open and close the valve, which requires a complex and annoying movement of a user's fingers.

It is an object of the invention to provide a haemostatic valve which can be easily and quickly operated and which is nevertheless reliable.

A haemostatic valve according to an embodiment may include a valve housing receiving an intravascular device, a first slider arranged on the valve housing moveable in a first direction, and a second slider received in the valve housing moveable in a second direction and elastically biased, the first slider being operatively connected to the second slider via a transmission mechanism so that movement of the first slider towards along the first direction causes the second slider to move away from an elastic valve member along the second direction, and the elastic valve member arranged between and compressible by the second slider, the elastic valve member receiving the intravascular device and compressing the intravascular device when being compressed by the second slider.

A haemostatic valve according to another embodiment may include a valve housing receiving an intravascular device, a pair of first sliders arranged on the valve housing opposite to each other and slidably moveable towards and away from each other in a first direction, and a pair of second sliders received in the valve housing and arranged opposite to each other and between the first sliders and slidably moveable towards and away from each other in a second direction and elastically biased towards each other, the first sliders being operatively connected to the second sliders via a transmission mechanism so that movement of the first sliders towards each other along the first direction causes the second sliders to move away from each other along the second direction, and an elastic valve member arranged between and compressible by the second sliders, the elastic valve member receiving the intravascular device and compressing the intravascular device when being compressed by the movement of the second sliders towards each other.

A haemostatic valve according to various embodiments allows for an easy handling and intuitive operation thereof. For example, for opening the valve a single unidirectional push motion of the first sliders along the first direction towards each other is sufficient; due to the construction of the haemostatic valve according to various embodiments, the haemostatic valve closes by itself when releasing the pushing force of the first sliders. For opening the valve, the first sliders are simply pushed towards each other along the first direction with a user's thumb and forefinger, that is, the haemostatic valve is ergonomically designed and utilizes a system that is more natural to be adjusted with the thumb and forefinger. Since the valve mechanism, which may be opened or closed numerous times during its use, requires the user only to simply push the first sliders towards each other to open the haemostatic valve, the valve eliminates any twisting motion using the forefinger and thumb. This twisting motion, which is currently used in conventional models, is a more complex muscle movement of the hand and prone to ‘repetitive’ injury. The valve closes on its own when the pushing force of the thumb and forefinger is released. Accordingly, only a partial release of the pushing force leads to a mere partial closure of the valve. This means, the haemostatic valve according to the various embodiments can provide for various distinct operating positions, namely: complete opening, controlled opening/closing and complete closure.

The intravascular device, for which the haemostatic valve is intended, can be any elongated operating instrument/device such as a guide wire or a catheter such as a balloon catheter or dilatation catheter. In operation, the intravascular device extends through the haemostatic valve, through the Y-Adapter connected with the haemostatic valve and into a blood vessel of a patient.

In an embodiment, the haemostatic valve can be used to fix the intravascular device into place so that forward and backward motion of the intravascular device within/relative to the haemostatic valve and Y-Adaptor (and thus within the vessel) can be prevented.

The haemostatic valve can be used, for example, to circumferentially seal the intravascular device in order to prevent leakage of blood out of the patient.

According to an embodiment, the haemostatic valve can be used to squeeze/release the intravascular device (such as a catheter) in order to control (e.g. prevent/allow) passage of a fluid there through. In this case, movement of the second sliders (in response to movement of the first sliders) causes constriction of the cross-section of the intravascular device.

The transmission mechanism may include a ramp-shaped section so that movement of the first slider along the first direction causes the second slider to move away the elastic valve member along the second direction.

In another embodiment, the transmission mechanism may include a ramp-shaped section so that movement of the first sliders along the first direction causes the second sliders to move away the elastic valve member along the second direction.

The transmission mechanism connecting the first and the second sliders (e.g. via the ramp-shaped section) may translate the pushing forces onto the first sliders along the first direction (e.g. in horizontal direction) into a motion of the second sliders along the second direction (e.g. in vertical direction) and against the elastic valve member. Twisting motion onto the intravascular device extending through the elastic valve member caused by twisting motion of the second sliders may be prevented by at least one guide arranged next to the second sliders and provided in the valve housing.

According to an embodiment, the transmission mechanism may include a ramp-shaped section and an engagement section engaged therewith. One of the first and second sliders may include a ramp and the other one may include the engagement section.

According to a further embodiment, the transmission mechanism may include a ramp-shaped section in form of two ramps. One of the first and second sliders may include a first ramp and the other one may include a second ramp corresponding to the first ramp.

The ramp-shaped section may be integrally provided on the first sliders and/or the second sliders. Further, the ramp-shaped section may be a triangle-shaped groove provided on the first sliders and/or the second sliders. If an engagement section is used, it may be provided in form of a fixedly or rotatably arranged ball or a rotatably arranged roller provided on an end portion or middle portion of the corresponding sliders. However, the engagement section may also have any other appropriate shape, for example a curved shape, allowing a sliding or rolling cam engagement between the ramp-shaped section and the engagement section.

In the open position of the valve the elastic valve member may be arranged/captured within the valve housing with the second sliders being biased against the valve housing, that is, the second sliders may be arranged within the valve housing so as to compress/deform the elastic valve member without any force acting onto the first sliders such that the valve is in its closed position. By movement of the second sliders (in response to movement of the first sliders) the amount of compression/deformation of the elastic valve member is decreased. By increasing the amount of compression/deformation of the elastic valve member by the second sliders, the intravascular device is fixed and/or sealed and/or squeezed.

According to an embodiment, the biasing of the second sliders against the valve housing is provided by coupling (e.g. fixing) the second sliders via a resilient member to the valve housing. The resilient member may be made of metal or plastic, and may be chosen from the group comprising a coil spring, a leaf spring, a compression spring, and a rubber device. The resilient member may be coupled (e.g. fixed) to the valve housing a nut which may be screwed in the valve housing to different positions so as to provide different pushing forces (up to 3 kg) onto the second sliders and, hence, different compression forces onto the elastic valve member.

In an embodiment, the elastic valve member may be made of rubber such as silicon rubber. However, other appropriate elastic materials are also possible for the elastic valve member. The elastic valve member may include a through hole through which the intravascular device extends. The elastic valve member may have, for example, the shape of a hollow cylinder. The elastic valve member may be circumferentially surrounded as a whole by the valve housing so that deformation of the elastic valve member (in response to movement of the second sliders) leads to corresponding constriction of the through passage/hole (through passage is closed/constricted by Poisson Effect), thereby fixing/sealing/squeezing the intravascular device.

According to an embodiment, the valve housing is provided in form of a hollow cylinder having a central axis along which the intravascular device extends, and a circumference with one or more openings (e.g. four openings arranged at 90°, 180°, 270° and 360°, in other words arranged at an angle distance of about 90° from the respective adjacent opening(s)), wherein each opening corresponds (e.g. alternately) to one of the first and second sliders. An opening corresponding to one of the second sliders may be formed to receive the nut for fixing the resilient member, whereas an opening corresponding to one of the first sliders may be formed to directly receive the respective first slider. Such an opening corresponding to the first sliders may have a rectangular cross-section. The first sliders may have a stopper at an end outside the valve housing and a limiting lever inside the valve housing, the stopper and the limiting lever restricting the movement of the first sliders through the valve housing.

The ramp-shaped section may be provided on the first sliders by means of two opposing first ramps which form a tapered, V-shaped section wherein the first ramps are tapered from the stopper to the limiting lever. The tapering angle of each ramp may be in the range between 5° and 30°. According to an embodiment, the tapering angle of each ramp is in the range between 10° and 20°, and may be chosen to be 15°.

According to the various embodiments, the first and second sliders may be formed from a synthetic polymeric material, for example, polycarbonate (PC), polyethylene (PE), polymethyl methacrylate (PMMA), polyvinyl chloride (PVC), or a combination thereof. The elastic valve member may be formed from a biocompatible elastomer, for example, silicone, polyurethane (PU), or a combination thereof.

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

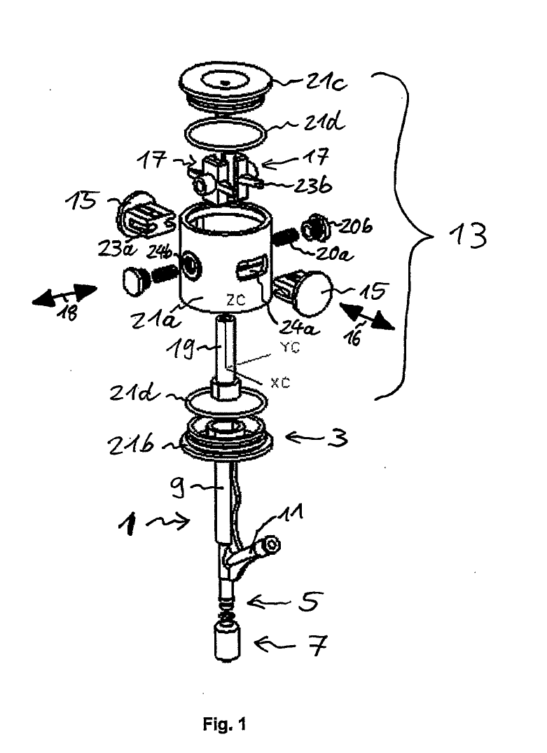

FIG. 1 shows an exploded view of a Y-adapter having a haemostatic valve according to an embodiment.

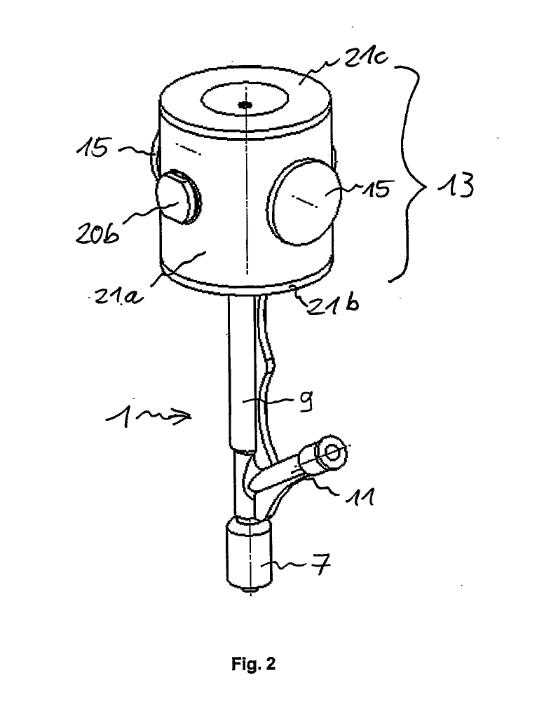

FIG. 2 shows a view of the Y-adapter having the haemostatic valve according to the embodiment in a mounted state.

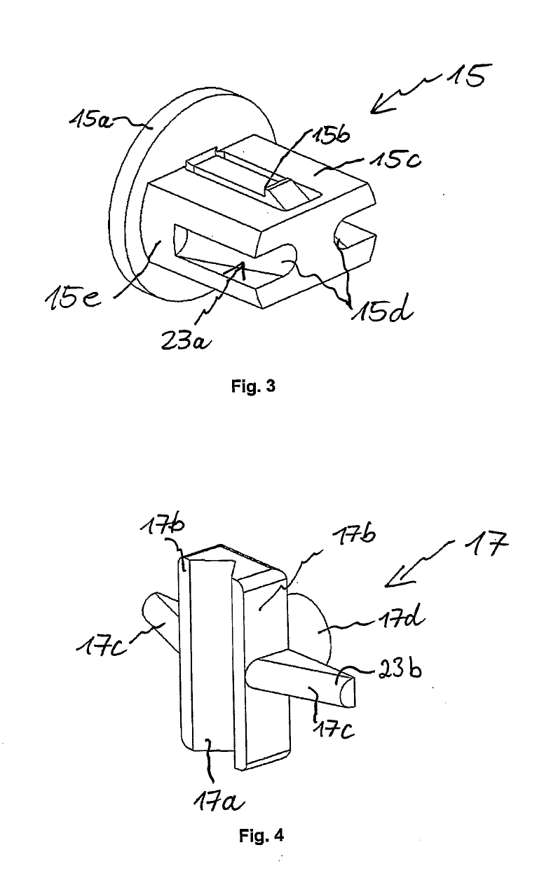

FIG. 3 shows an enlarged view of one of the first sliders.

FIG. 4 shows an enlarged view of one of the second sliders.

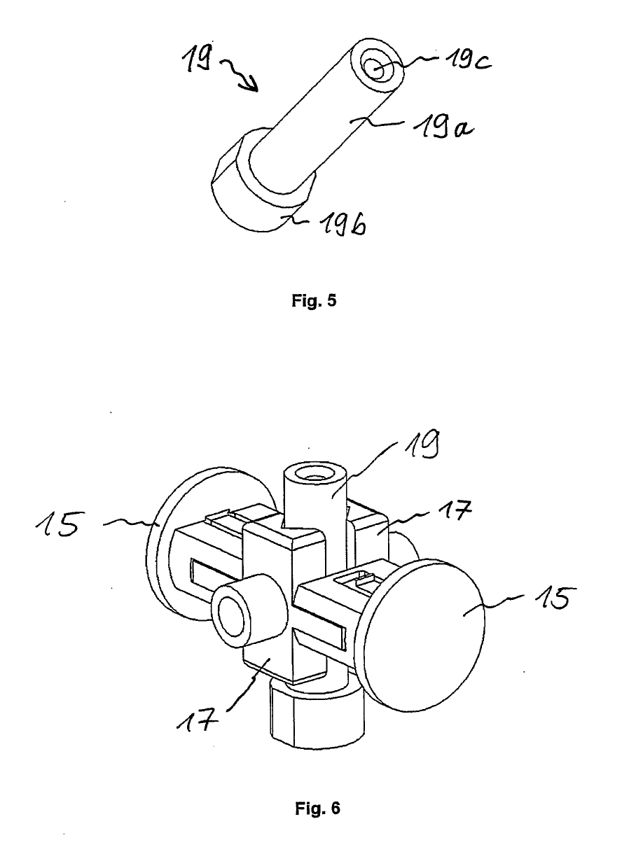

FIG. 5 shows an enlarged view of the elastic valve member.

FIG. 6 shows an enlarged view of the elastic valve member surrounded by the first and second sliders in a mounted state.

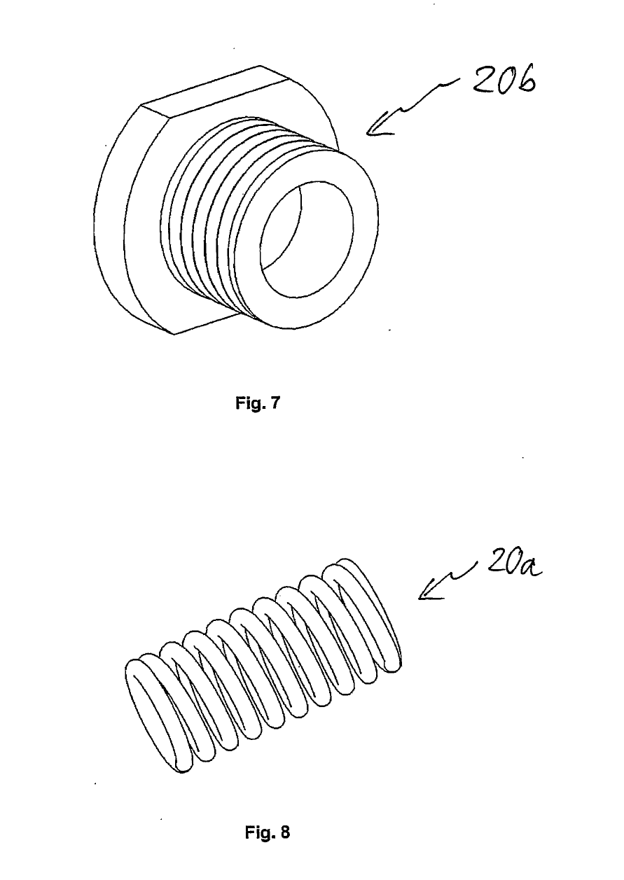

FIG. 7 shows an enlarged view of the nut.

FIG. 8 shows an enlarged view of the compression spring.

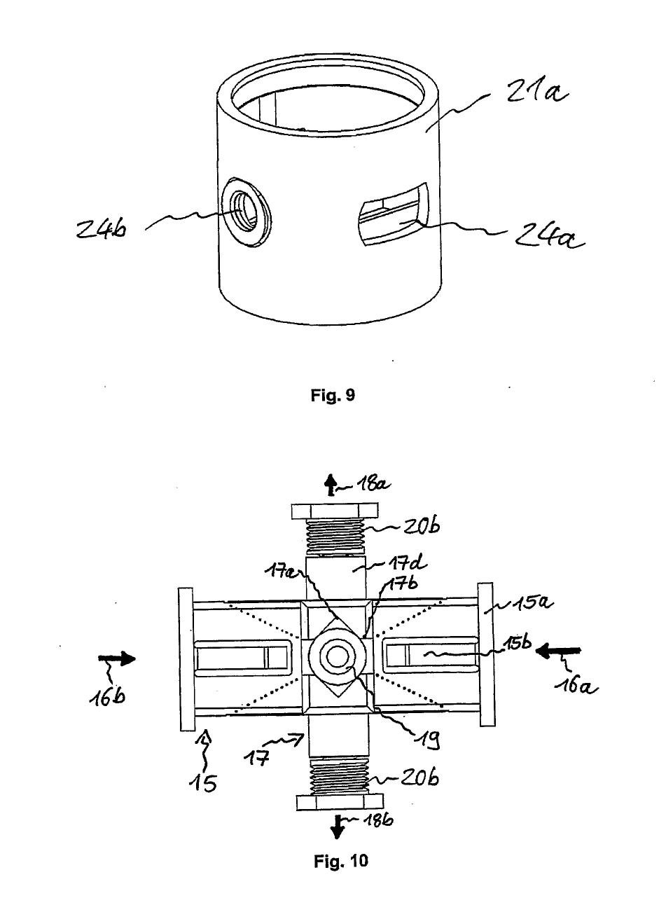

FIG. 9 shows an enlarged view of the main valve housing.

FIG. 10 shows a top view showing the interaction of the first sliders, the second sliders and the elastic valve member of the haemostatic valve in an actuated state, i.e., when a user acts onto the first sliders.

FIG. 11 shows a top view showing the interaction of the first sliders, the second sliders and the elastic valve member of the haemostatic valve in a released state, i.e., when a user does not act onto the first sliders.

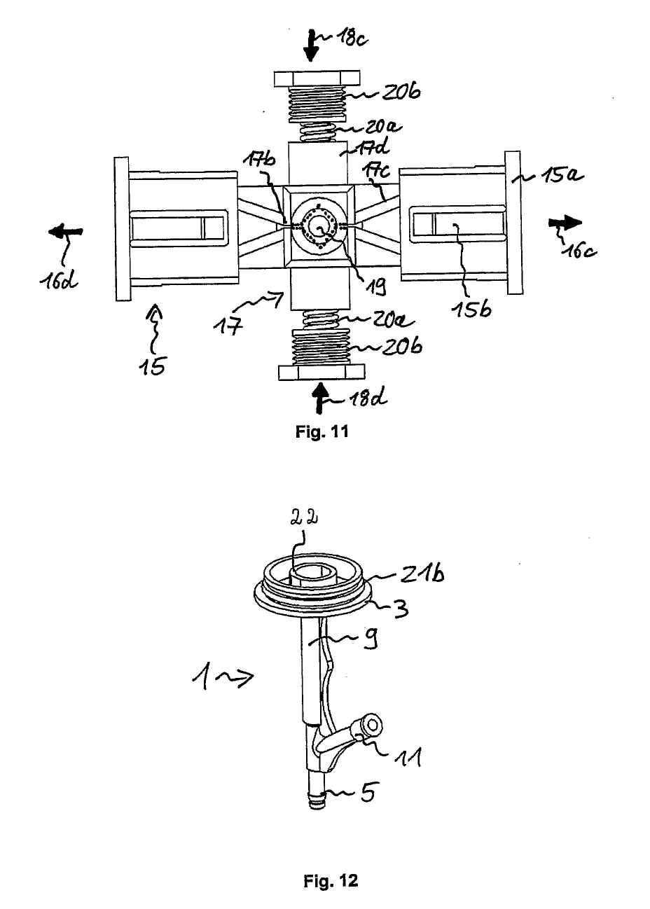

FIG. 12 shows an enlarged view of the Y-adapter.

FIG. 13 shows an enlarged view of the upper valve cover.

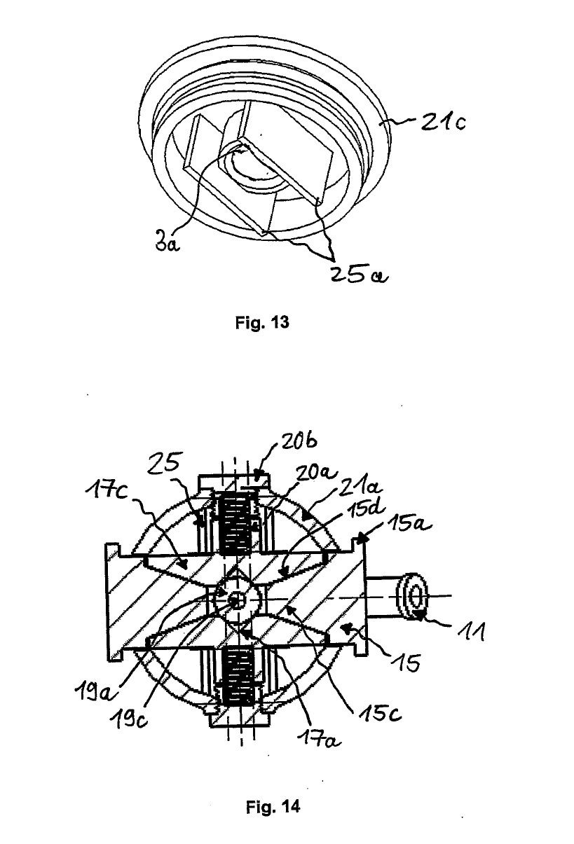

FIG. 14 shows a cross-sectional view of the haemostatic valve according to an embodiment taken perpendicular to the longitudinal axis of the haemostatic valve.

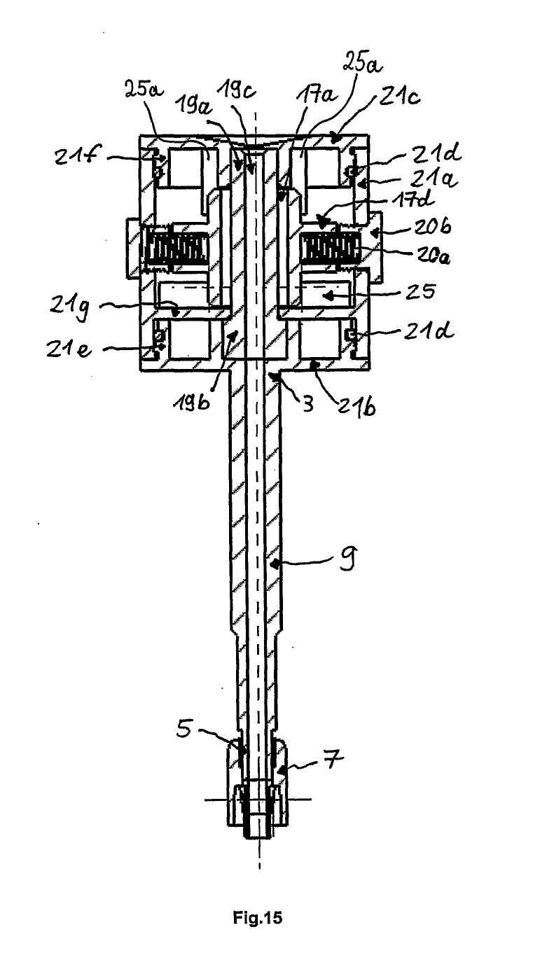

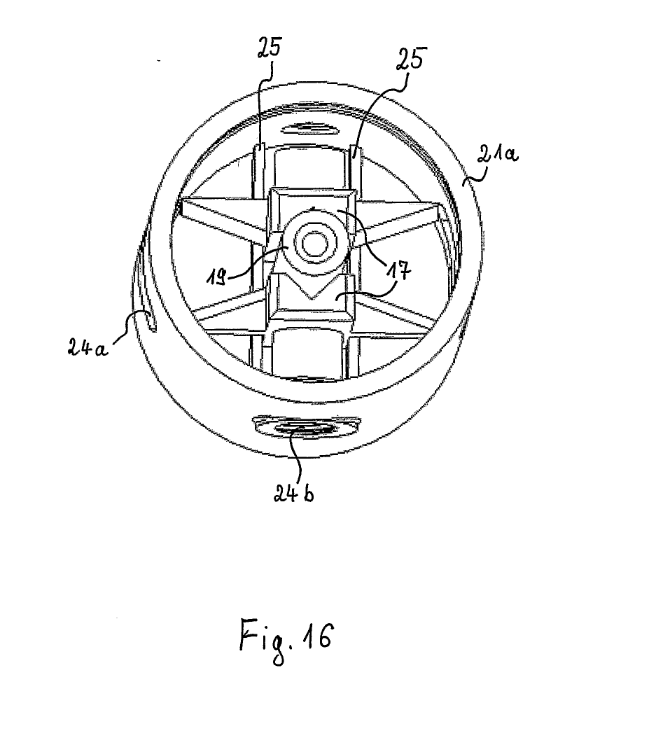

FIG. 15 shows a cross-sectional view of the haemostatic valve according to an embodiment taken along the longitudinal axis of the haemostatic valve.

FIG. 16 shows a top view of the main valve housing according to an embodiment.

FIG. 1 shows an exploded view of a Y-adapter 1 with a haemostatic valve 13 according to an embodiment.

The Y-adapter 1 (see also FIG. 12) has a proximal end 3 for receiving an intravascular device (not shown) and a distal end 5 to be connected to a guide catheter (not shown) by means of a Luer-lock mechanism 7. The Y-adapter 1 includes an elongated tubular main body 9 and an elongated tubular side arm 11 branching off from a middle portion or distal portion of the main body 9. In operation, the intravascular device such as a guide wire or a balloon catheter can be inserted into main body 9, and side arm 11 may be used to deliver/inject a fluid into the patient such as a saline solution or a contrast media. In another embodiment, the Y-adapter 1 can be provided with a plurality of side arms 11 branching-off from the main body 9. Furthermore, the Y-adapter 1 may include an elastic valve member receiving portion 22 having e.g. a hollow cylindrical shape configured to receive along its outer circumference the elastic valve member 19.

Now again with reference to FIG. 1, a haemostatic valve 13 is provided at the proximal end 3 of the Y-adapter 1. The haemostatic valve 13 includes a pair of first sliders 15, a pair of second sliders 17, an elastic valve member 19 and a valve housing 21 which in the present embodiment includes a main valve housing 21a in form of a hollow cylinder, an upper valve cover 21c and a lower valve cover 21b integrally formed with the Y-adapter 1. For sealing the valve housing 21, each an O-ring seal 21d is provided between the main valve housing 21a and the lower valve cover 21b, and between the main valve housing 21a and the upper valve cover 21c. In an alternative embodiment, the sealing may be provided using ultrasonic sealing. The upper and lower valve covers 21b, 21c are each provided with a central hole for leading the intravascular device there through. For guiding the second sliders 17 and preventing twisting motion of the second sliders 17, the upper valve cover 21c may include two guides 25a (see FIG. 13) and the main valve housing 21a also may include a guiding structure, e.g. in the form of one or more guiding ribs 25 guiding the second sliders 17 (see FIG. 16).

The first sliders 15 and the second sliders 17 are respectively received in the valve housing 21 so as to be respectively moveable in only one direction in a guided manner, wherein the first sliders 15 and the second sliders 17 are moveable in different directions. For example, the first sliders 15 may be moveable along (only) a first direction 16 and the second sliders 17 are moveable along (only) a second direction 18, wherein the first and second directions 16, 18 are substantially perpendicular to each other. The first sliders 15 may be arranged opposite each other on the main valve housing 21a, and the second sliders 17 are arranged opposite each other between the first sliders 15 inside the valve housing 21. Each first slider 15 is movably secured to the valve housing 21 by passing it through a corresponding first opening 24a in the main valve housing 21a, and each second slider 17 is movably fixed to the valve housing 21 by a resilient member 20a and a nut 20b screwed into a corresponding second opening 24b in the main valve housing 21a. This means, the first and second openings 24a, 24b are arranged alternately at the angles of 90°, 180°, 270° and 360° in the circumferential sidewall of the main valve housing 21a.

It should be mentioned that in an alternative embodiment, only exactly one first slider and/or only exactly one second slider may be provided configured to provide the similar function as the first sliders 15 and the second sliders 17.

In another embodiment, the second sliders 17 may be fixed (in general, coupled) via the respective resilient members 20a directly to the circumferential sidewall of the main valve housing 21a without the need of nuts 20b screwed into corresponding second openings 24b in the main valve housing 21a. This means, in this alternative embodiment only two opposing first openings 24a are provided in the circumferential sidewall of the main valve housing 21a.

The first sliders 15 and the second sliders 17 are connected via a transmission mechanism which translates the motion of the first sliders 15 along the first direction 16 into a motion of the second sliders 17 along the second direction 18. In an embodiment, the transmission mechanism includes a first ramp-shaped section 23a provided on the first sliders 15 and a second ramp-shaped section 23b provided on the second sliders 17 (see also FIG. 3 and FIG. 4). The first and second ramp-shaped sections 23a, 23b engage with each other with the first ramp-shaped section 23a forming a cam or drive section and with the second ramp-shaped section 23b forming an engagement or driven section. The amount and direction of inclination is substantially the same for each ramp-shaped section 23a, 23b. In an embodiment, the ramp-shaped sections 23a, 23b are integrally formed with the first and second sliders 15, 17. The ramp-shaped sections 23a, 23b are formed so as to mate with each other so that movement of the first sliders 15 along the first direction 16 causes the second sliders 17 to move along the second direction 18.

In another embodiment not shown herein, the transmission mechanism may include only one ramp-shaped section. For example, the ramp-shaped section may be integrally provided on the first sliders 15 as shown in FIG. 3, and a ball (not shown) acting as an engagement section may be integrally provided on an end portion or middle portion of the second sliders 17.

The at least one ramp-shaped section of the transmission mechanism may have such an inclination so as to provide a self-locking/self-impeding transmission mechanism so that the second sliders 17 can be moved only by means of correspondingly moving the first sliders 15 (towards or away from each other) but neither by the elastic force of the elastic valve member 19 (which, however, has an impact on the compression force; the higher the stiffness of the material of the elastic valve member 19, the higher the compression force of the second sliders 17 needs to be to close the aperture of the elastic valve member 19) nor by the blood pressure acting on the elastic valve member 19.

In alternative embodiments, all of the respective portions of the haemostatic valve 13 may be integrally formed. In an alternative embodiment, only some of the portions of the haemostatic valve 13 may be integrally formed. Furthermore, by way of example, the main valve housing 21a may be one integrally formed part or may for example include a plurality of e.g. two parts, e.g. two symmetric parts being separated along the main direction of the cylindrical main valve housing 21a. In this case, the two parts would be provided with respective engaging members for being mechanically coupled together to form the main valve housing 21a, for example. Furthermore, in this case, the upper valve cover 21c may also be split in a corresponding manner. Further, in this case each part of the two parts of the upper valve cover 21c may be integrally formed together with the respective one of the two parts of the main valve housing 21a.

FIG. 2 shows a view of the Y-adapter 1 having the haemostatic valve 13 according to an embodiment in a mounted state.

As can be seen in FIG. 3, each first slider 15 of an embodiment is formed as an ashlar-shaped element having a rectangular cross-section, integrally formed with a circular stopper 15a at one end thereof, and including a limiting lever 15b on a middle portion 15c at another end thereof, and two triangle-shaped grooves 15d at two opposing sidewalls 15e in the middle portion 15c which form the ramp-shaped section 23a. The outsides of the circular stoppers 15a allow pushing of the first sliders 15 with a user's thumb and forefinger. To this end, the outsides of the circular stoppers 15a may be provided with grooves or any other suitable pattern for increasing the grip between the outsides of the circular stoppers 15a and the user's thumb and forefinger. Each triangle-shaped groove 15d provides a first ramp for the ramp-shaped section 23a, and the two triangle-shaped grooves 15d together form a tapered, V-shaped section which tapers from the stopper 15a in direction to the limiting lever 15b. The stopper 15a and the corresponding limiting lever 15b provide limited movement of the respective first slider 15 within the corresponding first opening 24a through the main valve housing 21a. The triangle-shaped grooves 15d are inclined with respect to the sidewalls 15e of the middle portion 15c at an angle of 15°. However, other angles within the range of 5° to 30° are also possible. Other embodiments may use angles in the range of between 10° and 20°.

As can be seen in FIG. 4, each second slider 17 of the embodiment is a brake shoe-like element having a V-shaped groove 17a sandwiched between two opposing sidewalls 17b, the V-shaped groove 17a extending along the longitudinal axis of the second slider 17, two protrusions 17c extending in opposing directions out from the two sidewalls 17b, and a holder 17d. Each protrusion 17c forms a second ramp, the two second ramps together forming the ramp-shaped section 23b. The two protrusions 17c are engaged with corresponding triangle-shaped grooves 15d in order to form the transmission mechanism of the haemostatic valve 13. In operation, the intravascular device (not shown) may be inserted into the elastic valve member 19 extending along and compressed by the opposing V-shaped grooves 17a of the second sliders 17.

For example, the first and second sliders 15, 17 may be made of a suitable generally non-elastic synthetic material, for example a synthetic polymeric material such as polycarbonate (PC), polyethylene (PE), polymethyl methacrylate (PMMA), polyvinyl chloride (PVC), or a combination thereof.

FIG. 5 shows an enlarged view of the elastic valve member 19. In an embodiment, the elastic valve member 19 may include an elongated cylinder 19a and a cylindrical swelling 19b at one end thereof, and having a through hole 19c. The through hole 19c may have a diameter which is slightly larger than that of the intravascular device (not shown) for easily receiving the intravascular device. The elongated cylinder 19a is placed between the opposing V-shaped grooves 17a of the second sliders 17 and the cylindrical swelling 19b prevents the elastic valve member 19 from undesired slipping out of the second sliders 17. The elastic valve member 19 may be made of a suitable, generally known elastic material, e.g., biocompatible elastomers (such as silicon rubber and polyurethane), having suitable deformation characteristics.

FIG. 6 shows the elastic valve member 19 surrounded by the first and second sliders 15, 17 in a mounted state. For a detailed description of the shown parts, please refer to the descriptions of FIG. 3 to FIG. 5.

FIG. 7 shows an example of the nut 20b used for fixing the resilient member 20a at the main valve housing 21a by means of screwing the nut 20b into the main valve housing 21a. A variation of the amount of threads used for fixing the resilient member 20a at the main valve housing 21a enables a variation of the compression force onto the second sliders 17. Generally, a compression force of about 3 kg F is desired. Please note that the nut 20b may be of any commonly known configuration such that a detailed description thereof is omitted here. Further, other embodiments of the haemostatic valve 13 of the present invention may also be designed without the need of the nut 20b.

FIG. 8 shows an example of the resilient member 20a used for resiliently fixing the second sliders 17 at the main valve housing 21a. The shown example of the resilient member 20a is a coil spring made of metal or plastics. Other examples for the resilient member 20a are leaf springs, compression springs and suitably formed rubber devices. The resilient member 20a is responsible for enabling the user to operate the haemostatic valve 13 only with thumb and forefinger. When the user releases the pressing force onto the first sliders 15, the elastic valve member 19 is compressed via the second sliders 17 caused by the resilient members 20a.

FIG. 9 shows the main valve housing 21a of the embodiment of the haemostatic valve 13 shown in FIG. 1. Due to the shown orientation of the main valve housing 21a, one can see on the right side one first opening 24a for receiving one first slider 15, and on the left side one second opening 24b for receiving the nut 20a holding the corresponding second slider 17 via the resilient member 20b. The main valve housing 21a is completed to the valve housing 21 by means of the lower valve cover 21b and the upper valve cover 21c. The valve housing 21 may be made of any generally known suitable non-elastic synthetic material.

The interaction of the elements of the illustrative embodiment of the haemostatic valve 13 shown in FIG. 1 is now described with reference to FIG. 10 and FIG. 11. FIG. 10 shows a top view onto the actuated state of the elements, i.e., when a user has pressed the first sliders towards each other. FIG. 11 shows a top view onto the released (or idle) state of the elements, i.e., when a user has released the first sliders resulting in a maximum distance between the first sliders.

As shown in FIG. 10, in operation, a motion of the first sliders 15 towards each other in the first direction 16, which means moving the right first slider 15 into a first sub-direction 16a towards the left first slider 15 and moving the left first slider 15 into a second sub-direction 16b towards the right first slider 15 by, the user's thumb and forefinger, wherein the first and second sub-directions 16a, 16b are sub-directions of the first direction 16, is translated into a motion of the second sliders 17 away from each other in third and fourth sub-directions 18a, 18b via the transmission mechanism, wherein the third and fourth sub-directions 18a, 18b are sub-directions of the second direction 18. This motion of the second sliders 17 away from each other is received by the elastic valve element 19 whose deformation by the second sliders 17 during the idle state is released in accordance with this movement of the second sliders 17 away from each other. The movement of the top second slider 17 in the third sub-direction 18a away from the bottom second slider 17 and the movement of the bottom second slider 17 in the fourth sub-direction 18b away from the top second slider 17 causes the V-shaped grooves 17a of the second sliders 17 to move away from each other and the resilient members 20a to be compressed since the nuts 20b are stationary fixed in the main valve housing 21a in the mounted state. Alternatively, the resilient members 20a may be compressed by the wall of the main valve housing 21a itself, in which case the nuts 20b may be omitted. In this actuated state, the stoppers 15a of the first sliders 15 limit the movement of the first sliders 15 in the first and second sub-directions 16a, 16b, respectively, due to the limited opening of the first openings 24a. Please note that in the actuated state the protrusions 17c are not visible in the shown top view; however, the protrusions 17c are illustrated in FIG. 10 as dotted lines for increasing the clarity.

As shown in FIG. 11, in idle state when the user releases the compression force caused by his/her thumb and forefinger onto the first sliders 15, the first sliders 15 return into a position where they have a maximum possible distance from each other. This means, the right first slider 15 is moved into a fifth sub-direction 16c away from the left first slider 15 and the left first slider 15 is moved into a sixth sub-direction 16d away from the right first slider 15, wherein the fifth and sixth sub-directions 16c, 16d are sub-directions of the first direction 16. This movement of the second sliders 17 towards each other in seventh and eighth sub-directions 18c, 18d, wherein the seventh and eighth sub-directions 18c, 18d are sub-directions of the second direction 18, is translated via the transmission mechanism into a motion of the first sliders 15 away from each other. This motion of the second sliders 17 towards each other is transmitted by the protrusions 17c and, hence, received by the elastic valve element 19 by deforming/compressing it by the V-shaped grooves 17a in accordance with this movement of the second sliders 17 towards each other. Although the V-shaped grooves 17a are covered in top view by material of the elastic valve member 19, the borders of the V-shaped grooves 17a are illustrated here by dotted lines for increasing clarity. The movement of the top second slider 17 in the seventh sub-direction 18c towards the bottom second slider 17 and the movement of the bottom second slider 17 in the eighth sub-direction 18d towards the top second slider 17 are caused by releasing the compressed resilient members 20a. In this idle state, the limiting levers 15b of the first sliders 15 limit the movement of the first sliders 15 in the seventh and eighth sub-directions 16c, 16d, respectively, due to the fact that the lever hooks of the limiting levers 15b engage with the first openings 24a.

According to an embodiment, the deformation/compression of the elastic valve member 19 during the idle state is used to fix, seal and/or squeeze the intravascular device, whereas releasing the elastic valve member 19 is used during inserting, adjusting and/or removing the intravascular device.

Thus, by moving the second sliders 17 towards each other (automatically caused by releasing the first sliders 15 due to the resilient members 20a), the diameter of the through hole 19c can be reduced, thereby fixing and circumferentially sealing the intravascular device extending through the through hole 19c. By pressing only slightly onto the first sliders 15, the constriction of the cross-section of the intravascular device (such as a catheter) is slightly decreased, thereby enabling, e.g., an exact control of fluid flow within/through the intravascular device.

As shown in FIG. 13, the upper valve cover 21c has the form of a cap and includes at its center a receiving portion 3a to receive the elastic valve member 19. Tangentially adjacent the receiving portion 3a, there are provided two parallel guides 25a sandwiching the receiving portion 3a. Each guide 25a has the form of a wall extending out of, but integrally formed with the upper valve cover 21c. In the mounted state, the guides 25a extend from the upper valve cover 21c inside the main valve housing 21a towards the main valve housing 21a. The guides 25a serve for preventing the second sliders 17 from twisting. Please note that the guides 25a may also be arranged differently and/or may have different forms in other embodiments.

FIG. 14 is a cross-sectional view of the haemostatic valve 13 taken in a perpendicular plane to the longitudinal axis of the elongated cylinder 19a of the elastic valve member 19. In this view, the tapered, V-shaped section of the middle portion 15c of the first sliders 15 formed by the two triangle-shaped grooves 15d can clearly be seen. Further, in this view, the two first sliders 15 are forced towards each other resulting in a separation of the second sliders 17 causing a release of the elongated cylinder 19a of the elastic valve member 19. Additionally, the guides 25 for preventing twisting of the second sliders 17 can be seen at the bottom of the valve housing 21. Below the bottom of the valve housing 21, on the right side in FIG. 14, the elongated tubular side arm 11 of the Y-adapter 1 can be seen.

FIG. 15 is a cross-sectional view of the haemostatic valve 13 and the Y-adapter 1 taken along the longitudinal axis of the elongated cylinder 19a of the elastic valve member 19 through the second sliders 17. As can be taken from this view, each of the upper and lower valve covers 21b, 21c comprises an integrally formed protuberant wall 21e, 21f, respectively. The protuberant walls 21e, 21f overlap with the main valve housing 21a and serve together with the O-ring seals 21d for a sealed fixation of the upper and lower valve covers 21b, 21c to the main valve housing 21a. Accordingly, the protuberant walls 21e, 21f have a radius with respect to the central axis of the main valve housing 21a which is slightly smaller than the radius of the main valve housing 21a itself.

The embodiment of FIG. 15 shows that the guides 25 are mounted onto an intermediate valve bottom 21g which is parallel to the lower valve cover 21b but extends centrally from the main valve housing 21a directly above the protuberant wall 21e. It is clear that the intermediate valve bottom 21g must have a central opening for accommodating the elongated cylinder 19a of the elastic valve member 19 there through.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A haemostatic valve comprising:
a valve housing receiving an intravascular device,
a first slider arranged on the valve housing moveable in a first direction, and
a second slider received in the valve housing moveable in a second direction and elastically biased,
the first slider being operatively connected to the second slider via a transmission mechanism so that movement of the first slider towards along the first direction causes the second slider to move away from an elastic valve member along the second direction, and
the elastic valve member arranged between and compressible by the second slider, the elastic valve member receiving the intravascular device and compressing the intravascular device when being compressed by the second slider.

2. The haemostatic valve according to claim 1,
wherein the transmission mechanism comprises a ramp-shaped section so that movement of the first slider along the first direction causes the second slider to move away the elastic valve member along the second direction.

3. A haemostatic valve comprising:
a valve housing receiving an intravascular device,
a pair of first sliders arranged on the valve housing opposite to each other and slidably moveable towards and away from each other in a first direction, and
a pair of second sliders received in the valve housing and arranged opposite to each other and between the first sliders and slidably moveable towards and away from each other in a second direction and elastically biased towards each other,
the first sliders being operatively connected to the second sliders via a transmission mechanism so that movement of the first sliders towards each other along the first direction causes the second sliders to move away from each other along the second direction, and
an elastic valve member arranged between and compressible by the second sliders, the elastic valve member receiving the intravascular device and compressing the intravascular device when being compressed by the movement of the second sliders towards each other.

4. The haemostatic valve according to claim 3,
wherein the transmission mechanism comprises a ramp-shaped section so that movement of the first sliders along the first direction causes the second sliders to move away the elastic valve member along the second direction.

5. The haemostatic valve according to claim 1,
wherein the first direction and the second direction are substantially perpendicular to each other.

6. The haemostatic valve according to claim 1,
wherein each first slider comprises at least one first ramp, each second slider comprises at least one second ramp, the first and second ramps forming the ramp-shaped section of the transmission mechanism, each first ramp being in operative contact with a corresponding one of the second ramps to form the transmission mechanism.

7. The haemostatic valve according to claim 1,
wherein each second slider is coupled to the valve housing via a resilient member.

8. The haemostatic valve according to claim 7,
wherein the resilient member is a resilient member selected from a group of resilient members consisting of: a coil spring, a leaf spring, a compression spring, and a rubber device.

9. The haemostatic valve according to claim 7,
wherein the resilient member is coupled to the valve housing by a nut so as to provide different compressing forces onto the elastic valve member.

10. The haemostatic valve according to claim 7,
wherein the resilient member is supported by the side wall of the valve housing and thereby pressed to the second slider with a predefined compressive force.

11. The haemostatic valve according to claim 1,
wherein a through hole is formed in each second slider through which the intravascular device extends.

12. The haemostatic valve according to claim 1,
wherein the elastic valve member comprises a through hole through which the intravascular device extends so that the elastic valve member is radially pressed against the intravascular device by the second slider along the whole circumference of the intravascular device.

13. The haemostatic valve according to claim 3,
wherein the valve housing is a hollow cylinder having in its circumference openings configured to receive the first slider and the second slider, respectively.

14. The haemostatic valve according to claim 13,
wherein the valve housing has in its circumference four openings being arranged at an angle of 90° apart from each other and configured to receive the first slider and the second slider, respectively.

15. The haemostatic valve according to claim 13,
wherein the hollow cylinder comprises a central axis, the intravascular device extending through the central axis.

16. The haemostatic valve according to claim 14,
wherein the first sliders extend through two opposing ones of the four openings, the first sliders having a substantially rectangular cross-section so that the corresponding two openings are substantially rectangular.

17. The haemostatic valve according to claim 1,
wherein each of the first slider comprises at its end outside the valve housing a stopper and inside the valve housing a limiting lever, the stopper and the limiting lever enabling limited movement of the first slider through the valve housing.

18. The haemostatic valve according to claim 17,
wherein each first slider comprises two opposing first ramps forming a tapered, V-shaped section along the rectangular cross-section of the first slider, the tapered, V-shaped section tapering from the stopper in direction to the limiting lever.

19. The haemostatic valve according to claim 1,
wherein the valve housing comprises at least one guide arranged next to the second slider, the at least one guide preventing twisting of the second slider.

20. A Y-adapter comprising a haemostatic valve according to claim 1.
